(19) United States
(12) Patent Application Publication
(10) Pub. No.: US 2023/0220356 A1
(43) Pub. Date: Jul. 13, 2023

(54) COMPOSITIONS AND METHODS FOR PRODUCING A VIRAL VACCINE WITH REDUCED PARTICLE SIZE

(71) Applicant: Seqirus UK Limited, Berkshire (GB)

(72) Inventors: Christopher DADD, Holly Springs, NC (US); Matthew SMITH, Holly Springs, NC (US); Debra LYDIARD, Holly Springs, NC (US)

(21) Appl. No.: 17/771,625

(22) PCT Filed: Nov. 6, 2020
(86) PCT No.: PCT/IB2020/000961

§ 371 Date: Apr. 25, 2022

Related U.S. Application Data
(60) Provisional application No. 62/931,909, filed on Nov. 7, 2019.

(51) Int. Cl. C12N 7/06

(52) U.S. Cl. CPC C12N 7/06; A61K 2039/5252

(57) ABSTRACT

Disclosed herein are methods and composition producing a viral vaccine with reduced particle size, particularly for use in the production of influenza virus vaccines.

### FIELD OF THE DISCLOSURE

Disclosed are compositions and methods for producing a viral vaccine with reduced particle size.

### BACKGROUND

The antigen in a viral vaccine can comprise purified components (e.g., a recombinant viral protein) or fragments of viruses modified to lose their virulence (e.g., an inactivated virus). For example, some influenza vaccines contain influenza virus that has been irreversibly inactivated by treatment with alkylating, thereby altering their viral nucleic acids or protein. Regardless of the antigenic component, all vaccines must undergo extensive processing and purification after production before they can be administered to patients. Purification often involves chromatography and filtration steps to remove undesired by-products generated from the production of the antigen, for example components of host cell, or chemicals added along the way.

Some influenza vaccines are produced by inactivating live viruses. Viruses may be grown in pathogen-free eggs or cultured cell lines, before they are harvested and purified from their host cells. The viruses may then be inactivated by heat or chemicals, and subsequently subjected to a splitting step, during which the viral particles are broken up to form smaller sub-virions. Depending on the conditions during splitting, sub-virions may not achieve monodispersity and/or the desired size, which may affect downstream purification processes. For example, salt concentrations during splitting may provide inadequate shielding of electrostatic charges, causing sub-virion units to aggregate and form larger particles. These aggregates may then compromise purification columns, and foul filters leading to reduced throughput, lower overall yields, and inability to manufacture the vaccine. Thus, improved processes are needed that can increase filter throughput and yield, especially during the ramp up in production of seasonal influenza vaccines or during a pandemic.

The inventors surprisingly found that the size of split sub-virions can be reduced by treating the particles with a reagent comprising at least one component selected from the group consisting of a non-ionic surfactant, an ionic surfactant, and a salt, and wherein the at least one component is present in an amount effective to reduce particle size of the sub-virions. The disclosure provides methods and compositions that can be used to produce viral vaccines with reduced particle sizes. The disclosure also provides methods and compositions for splitting viral particles to form sub-virions, and contemplates their use in the production of influenza virus vaccines.

As the present disclosure shows, the size of sub-virion particles inversely correlates with filter throughput. Accordingly, strategies to increasing filter throughput and yield during viral vaccine production may include reducing sub-virion particle sizes through splitting, or maintaining ideal conditions throughout processing to prevent aggregation of sub-virions. This can be achieved by the methods and compositions of the invention including varying the conditions and relative amounts of the reagents (e.g., salt, surfactant) involved in the splitting step.

### SUMMARY

The disclosure provides methods and compositions for producing viral vaccines with reduced particle sizes and methods and compositions for splitting viral particles to form sub-virions, and contemplates their use in the production of influenza virus vaccines.

The present disclosure provides a method of producing a viral vaccine formulated in a sub-virion form, the method comprising the steps:
a. purifying viral particles from harvested cell culture;
b. inactivating and splitting the viral particles to produce sub-virions; and
c. purifying the sub-virions.
   wherein inactivating and splitting the viral particles of step b comprises treating the viral particles with a reagent comprising a non-ionic surfactant and an ionic surfactant, and wherein the non-ionic surfactant and the ionic surfactant are present in an amount effective to reduce particle size of the viral particles.

In some embodiments, the reagent further comprises a salt, wherein the salt is present in an amount effective to reduce particle size of the viral particles.

In some embodiments, the average hydrodynamic radius of the sub-virion ranges from 150 nm to 350 nm.

In some embodiments, the non-ionic surfactant is polysorbate 80. In some embodiments, the non-ionic surfactant comprises at least about 1.0 g/L polysorbate 80, e.g., at least about 1.5 g/L, at least about 2.0 g/L, or at least about 2.5 g/L. In some embodiments, the ionic surfactant comprises cetrimonium bromide (CTAB). In some embodiments, the ionic surfactant comprises about 1.25 g/L-3.0 g/L CTAB, e.g., about 1.5 g/L, about 2.0 g/L, about 2.5 g/L, or about 3.0 g/L. In some embodiments, the salt comprises sodium chloride (NaCl). In some embodiments, the salt comprises 0-200 mM NaCl, e.g., about 25 mM, about 50 mM, about 75 mM, about 100 mM, about 125 mM, about 150 mM, or about 175 mM. In some embodiments, the non-ionic surfactant comprises polysorbate 80, the ionic surfactant comprises CTAB, and the salt comprises sodium chloride NaCl.

In some embodiments of the method, purifying in step c comprises adsorption filtration. In some such embodiments, adsorption filtration throughput is at least about 50 L/m², e.g., at least about 100 L/m², at least about 150 L/m², at least about 200 L/m², at least about 200 L/m², at least about 300 L/m², or at least about 350 L/m². In some other embodiments, the particle size of the sub-virion is less than about 500 nm, e.g., less than about 400 nm, less than about 300 nm, less than about 200 nm, or less than about 150 nm. In some embodiments, the particle size of the sub-virion is the hydrodynamic radius. In some embodiments, the size of the sub-virion is measured by dynamic light scattering (DLS).

In some embodiments of the method, the viral particles are from the influenza virus. In some embodiments, the viral particles are from the influenza virus A strain.

The present disclosure also provides a method of reducing viral particle size, the method comprising treating the viral particles with a reagent comprising a non-ionic surfactant and an ionic surfactant, and wherein the non-ionic surfactant and an ionic surfactant are present in an amount effective to reduce particle size of the viral particles. In some embodiments, the reagent further comprises a salt, wherein the salt is present in an amount effective to reduce particle size of the viral particles

In some embodiments, the non-ionic surfactant comprises polysorbate 80. In some embodiments, the non-ionic surfactant comprises at least about 1.0 g/L polysorbate 80, e.g., at least about 1.5 g/L, at least about 2.0 g/L, or at least about 2.5 g/L. In some embodiments, ionic surfactant comprises cetrimonium bromide (CTAB). In some embodiments, the ionic surfactant comprises about 1.25 g/L-3.0 g/L CTAB, e.g., about 1.5 g/L, about 2.0 g/L, about 2.5 g/L, or about 3.0 g/L. In some embodiments, the salt comprises sodium chloride (NaCl). In some embodiments, the salt comprises 25-200 mM NaCl, e.g., about 50 mM, about 75 mM, about 100 mM, about 125 mM, about 150 mM, or about 175 mM. In some embodiments, the non-ionic surfactant comprises polysorbate 80, the ionic surfactant comprises CTAB, and the salt comprises sodium chloride NaCl.

In some embodiments, the method further comprises filtering the treated viral particles through an adsorption filter. In some embodiments, the throughput of the adsorption filter is at least about 50 L/m², e.g., at least about 100 L/m², at least about 150 L/m², at least about 200 L/m², at least about 250 L/m², at least about 300 L/m², or at least about 350 L/m². In some embodiments, the viral particle size is less than about 500 nm, e.g., less than about 400 nm, less than about 300 nm, less than about 200 nm, or less than about 150 nm. In some embodiments, the viral particle size is the hydrodynamic radius of the viral particle. In some embodiments, the viral particle size is measured by dynamic light scattering (DLS).

In some embodiments, the viral particles are from the influenza virus. In some embodiments, the viral particles are from the influenza virus A strain.

The present disclosure also provides a method of reducing viral particle size in an influenza virus purification process, the method comprising
a. purifying viral particles harvested from cell culture;
b. inactivating and splitting the viral particles to produce sub-virions; and
c. purifying the sub-virions.
   wherein activating and splitting the viral particles of step b comprises treating the viral particles with a reagent comprising a non-ionic surfactant and an ionic surfactant, and wherein the non-ionic surfactant and the ionic surfactant are present in an amount effective to reduce particle size of the viral particles.

The present disclosure also provides a method of producing a viral vaccine formulated in a sub-virion form, the method comprising the steps:
a. purifying viral particles from harvested cell culture;
b. inactivating and splitting the viral particles to produce sub-virions; and
c. purifying the sub-virions.
   wherein activating and splitting the viral particles of step b comprises treating the viral particles with a reagent comprising a non-ionic surfactant and an ionic surfactant, and wherein the non-ionic surfactant and the ionic surfactant are present in an amount effective to reduce particle size of the viral particles.

The present disclosure also provides a method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
   wherein inactivating and splitting the viral particles of step b comprises treating the viral particles with a reagent comprising a non-ionic surfactant and an ionic surfactant, and wherein the non-ionic surfactant and the ionic surfactant are present in an amount effective to reduce particle size of the viral particles.

In some embodiments, the reagent further comprises a salt, wherein the salt is present in an amount effective to reduce particle size of the viral particles.

In some embodiments, the non-ionic surfactant comprises polysorbate 80. In some embodiments, the non-ionic surfactant comprises at least about 1.0 g/L polysorbate 80, e.g., at least about 1.5 g/L, at least about 2.0 g/L, or at least about 2.5 g/L. In some embodiments, the salt comprises sodium chloride (NaCl). In some embodiments, the salt comprises 25-200 mM NaCl, e.g., about 50 mM, about 75 mM, about 100 mM, about 125 mM, about 150 mM, or about 175 mM.

In particular embodiments, the concentration of polysorbate 80 in the non-ionic surfactant comprises 0.3 g/L following inactivation of the viral particles and prior to splitting. In further embodiments, the concentration of polysorbate 80 in the non-ionic surfactant increases at a range of 0-2.2 g/L during splitting. Thus, potential polysorbate 80 concentrations during splitting may include 0.3 g/L, 1.4 g/L, and 2.5 g/L.

The present disclosure also provides a method of producing an influenza viral vaccine formulated comprising purified viral proteins, the method comprising the steps:
a. purifying viral particles from a harvested cell culture of MDCK cells;
b. inactivating and splitting the viral particles to produce sub-virions; and
c. purifying the sub-virions to produce purified viral proteins;
   wherein inactivating and splitting the viral particles of step b comprises treating the viral particles with a reagent comprising a non-ionic surfactant and a salt, and wherein the non-ionic surfactant and the salt are in an amount effective to reduce particle size of the viral particles.

The present disclosure also provides a method of producing an influenza viral vaccine formulated comprising purified viral proteins, the method comprising the steps:
a. purifying viral particles from a harvested cell culture of MDCK cells;
b. inactivating and splitting the viral particles to produce sub-virions; and
c. purifying the sub-virions to produce purified influenza viral proteins;
   wherein inactivating and splitting the viral particles of step b comprises treating the viral particles with a reagent comprising an ionic surfactant and a salt, and wherein the ionic surfactant and the salt are in an amount effective to reduce particle size of the viral particles.

The present disclosure also provides a method of producing an influenza viral vaccine formulated comprising purified viral proteins, the method comprising the steps:
a. purifying viral particles from a harvested cell culture of MDCK cells;
b. treating the purified viral particles with alkylating agent to inactivate the viral particles;
c. treating the inactivated viral particle with an ionic surfactant to split the inactivated viral particles to produce subvirions; and
d. purifying the sub-virions to produce purified influenza viral proteins;
   wherein inactivating and splitting the viral particles of step b and/or c further comprises treating the viral particles with a reagent comprising a non-ionic surfactant and an ionic surfactant, and wherein the non-ionic surfactant and the ionic surfactant are in an amount effective to reduce particle size of the viral particles.

The present disclosure also provides a method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
   wherein inactivating and splitting the viral particles of step b comprises treating the viral particles with a reagent comprising CTAB, NaCl, and polysorbate 80, in an amount effective to reduce particle size of the viral particles.

The present disclosure also provides a method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
   wherein inactivating and splitting the viral particles of step b comprises treating the viral particles with a reagent comprising 1.5-2.5 g/L CTAB, 0-150 mM NaCl, and 0-2.2 g/L polysorbate 80, in an amount effective to reduce particle size of the viral particles.

The present disclosure also provides a method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
   wherein inactivating and splitting the viral particles of step b comprises treating the viral particles with a reagent comprising 1.5-2.5 g/L of an ionic surfactant, 0-150 mM of a salt, and 0-2.2 g/L of a non-ionic surfactant, in an amount effective to reduce particle size of the viral particles.

In some embodiments, the ionic surfactant is CTAB. In some embodiments, the salt is NaCl. In some embodiments, the non-ionic surfactant is polysorbate 80.

### BRIEF DESCRIPTION OF FIGURES

FIGS. 1A-C: The average particle size of Influenza B/Victoria lineage sub-virions produced under different splitting conditions as measured by DLS. The relative amounts of CTAB, polysorbate 80, and NaCl were varied.

FIGS. 2A-C: The average particle size of Influenza A H3N2 subtype sub-virions produced under different splitting conditions as measured by DLS. The relative amounts of CTAB, polysorbate 80, and NaCl were varied.

FIGS. 3A-C: The average particle size of Influenza A H3N2 subtype sub-virions produced under different splitting conditions as measured by DLS. The relative amounts of CTAB, polysorbate 80, and NaCl were varied.

FIG. 4: The filter throughput of the adsorption filter during purification of the sub-virion as a function of particle size of the sub-virion. Shown are H1 N1 strains designated as IVR-180 and TT1384.

### DETAILED DESCRIPTION

The disclosed processes and compositions may be understood more readily by reference to the following detailed description taken in connection with the accompanying figures, which form a part of this disclosure.

Throughout this text, the descriptions refer to processes and compositions for making a viral vaccine. Where the disclosure discloses or claims a feature or embodiment associated with a process of making a viral vaccine, such a feature or embodiment is equally applicable to the viral vaccine. Likewise, where the disclosure discloses or claims a feature or embodiment associated with a viral vaccine, such a feature or embodiment is equally applicable to the process or compositions for making the viral vaccine. When a range of values is expressed, it includes embodiments using any particular value within the range. Further, reference to values stated in ranges includes each and every value within that range. When values are expressed as approximations by use of the antecedent “about” it will be understood that the particular value forms another embodiment. The use of “or” will mean “and/or” unless the specific context of its use dictates otherwise. All references cited herein are incorporated by reference in their entirety. Where a reference and the specification conflict, the specification will control. It is to be appreciated that certain features of the disclosed processes and compositions, which are, for clarity, disclosed herein in the context of separate embodiments, may also be provided in combination in a single embodiment. Conversely, various features of the disclosed processes and compositions that are, for brevity, disclosed in the context of a single embodiment, may also be provided separately or in any sub-combination.

As used herein, the singular forms “a,” “an,” and “the” include plural forms unless the context clearly dictates otherwise. The terms “including,” “includes,” “having,” “has,” “with,” or variants thereof used in either the detailed description and/or the claims, are intended to be inclusive in the same manner as the term “comprising.” The term “about” or “approximately,” when used in the context of numerical values and ranges, refers to values or ranges that approximate or are close to the recited values or ranges such that the embodiment may perform as intended, up to about plus or minus 10%, as is apparent to the skilled person from the teachings contained herein. In some embodiments, about means plus or minus 10% of a numerical amount.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition that “comprises,” “has” or “includes” one or more features is not limited to possessing only those one or more features and can cover other unlisted features. The use of any and all examples, or exemplary language (e.g., “such as”) provided with respect to certain embodiments herein is intended merely to better illuminate the present disclosure and does not pose a limitation on the scope of the present disclosure otherwise claimed.

The present disclosure provides a method of producing a viral vaccine formulated in a sub-virion form, the method comprising the steps:
a. purifying viral particles from harvested cell culture;
b. inactivating and splitting the viral particles to produce sub-virions; and
c. purifying the sub-virions;
   wherein inactivating and splitting the viral particles of step b comprises treating the viral particles with a reagent comprising at least one component selected from the group consisting of a non-ionic surfactant and an ionic surfactant, and wherein the non-ionic surfactant and the ionic surfactant are present in an amount effective to reduce particle size of the viral particles.

In some embodiments, the reagent further comprises a salt, wherein the salt is present in an amount effective to reduce particle size of the viral particles.

In some embodiments, the average hydrodynamic radius of the sub-virion ranges from 150 nm to 350 nm.

In some embodiments, the non-ionic surfactant is polysorbate 80. In some embodiments, the non-ionic surfactant comprises at least about 1.0 g/L polysorbate 80, e.g., at least about 1.5 g/L, at least about 2.0 g/L, or at least about 2.5 g/L. In some embodiments, the ionic surfactant comprises cetrimonium bromide (CTAB). In some embodiments, the ionic surfactant comprises about 1.25 g/L-3.0 g/L CTAB, e.g., about 1.5 g/L, about 2.0 g/L, about 2.5 g/L, or about 3.0 g/L. In some embodiments, the salt comprises sodium chloride (NaCl). In some embodiments, the salt comprises 0-200 mM NaCl, e.g., about 25 mM, about 50 mM, about 75 mM, about 100 mM, about 125 mM, about 150 mM, or about 175 mM. In some embodiments, the non-ionic surfactant comprises polysorbate 80, the ionic surfactant comprises CTAB, and the salt comprises sodium chloride NaCl.

In some embodiments of the method, purifying in step c comprises adsorption filtration. In some such embodiments, adsorption filtration throughput is at least about 50 L/m², e.g., at least about 100 L/m², at least about 150 L/m², at least about 200 L/m², at least about 200 L/m², at least about 300 L/m², or at least about 350 L/m². In some other embodiments, the particle size of the sub-virion is less than about 500 nm, e.g., less than about 400 nm, less than about 300 nm, less than about 200 nm, or less than about 150 nm. In some embodiments, the particle size of the sub-virion is the hydrodynamic radius. In some embodiments, the size of the sub-virion is measured by dynamic light scattering (DLS).

In some embodiments of the method, the viral particles are from the influenza virus. In some embodiments, the viral particles are from the influenza virus A strain.

The present disclosure also provides a method of reducing viral particle size, the method comprising treating the viral particles with a reagent comprising at least one component selected from the group consisting of a non-ionic surfactant and an ionic surfactant, and wherein the non-ionic surfactant and the ionic surfactant are present in an amount effective to reduce particle size of the viral particles.

In some embodiments, the non-ionic surfactant comprises polysorbate 80. In some embodiments, the non-ionic surfactant comprises at least about 1.0 g/L polysorbate 80, e.g., at least about 1.5 g/L, at least about 2.0 g/L, or at least about 2.5 g/L. In some embodiments, ionic surfactant comprises cetrimonium bromide (CTAB). In some embodiments, the ionic surfactant comprises about 1.25 g/L-3.0 g/L CTAB, e.g., about 1.5 g/L, about 2.0 g/L, about 2.5 g/L, or about 3.0 g/L. In some embodiments, the salt comprises sodium chloride (NaCl). In some embodiments, the salt comprises 25-200 mM NaCl, e.g., about 50 mM, about 75 mM, about 100 mM, about 125 mM, about 150 mM, or about 175 mM. In some embodiments, the non-ionic surfactant comprises polysorbate 80, the ionic surfactant comprises CTAB, and the salt comprises sodium chloride NaCl.

In some embodiments, the method further comprises filtering the treated viral particles through an adsorption filter. In some embodiments, the throughput of the adsorption filter is at least about 50 L/m², e.g., at least about 100 L/m², at least about 150 L/m², at least about 200 L/m², at least about 250 L/m², at least about 300 L/m², or at least about 350 L/m². In some embodiments, the viral particle size is less than about 500 nm, e.g., less than about 400 nm, less than about 300 nm, less than about 200 nm, or less than about 150 nm. In some embodiments, the viral particle size is the hydrodynamic radius of the viral particle. In some embodiments, the viral particle size is measured by dynamic light scattering (DLS).

In some embodiments, the viral particles are from the influenza virus. In some embodiments, the viral particles are from the influenza virus A strain.

The present disclosure also provides a method of reducing viral particle size in an influenza virus purification process, the method comprising
a. purifying viral particles harvested from cell culture;
b. inactivating and splitting the viral particles to produce sub-virions; and
c. purifying the sub-virions;
   wherein activating and splitting the viral particles of step b comprises treating the viral particles with a reagent comprising a non-ionic surfactant and an ionic surfactant, and wherein the non-ionic surfactant and the ionic surfactant are present in an amount effective to reduce particle size of the viral particles.

The present disclosure also provides a method of producing a viral vaccine formulated in a sub-virion form, the method comprising the steps:
a. purifying viral particles from harvested cell culture;
b. inactivating and splitting the viral particles to produce sub-virions; and
c. purifying the sub-virions;
   wherein activating and splitting the viral particles of step b comprises treating the viral particles with a reagent a non-ionic surfactant and an ionic surfactant, and wherein the non-ionic surfactant and the ionic surfactant are present in an amount effective to reduce particle size of the viral particles.

The present disclosure also provides a method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
   wherein inactivating and splitting the viral particles of step b comprises treating the viral particles with a reagent comprising a non-ionic surfactant and an ionic surfactant, and wherein the non-ionic surfactant and the ionic surfactant are present in an amount effective to reduce particle size of the viral particles.

In some embodiments, the reagent further comprises a salt, wherein the salt is present in an amount effective to reduce particle size of the viral particles. In some embodiments, the non-ionic surfactant comprises polysorbate 80. In some embodiments, the non-ionic surfactant comprises at least about 1.0 g/L polysorbate 80, e.g., at least about 1.5 g/L, at least about 2.0 g/L, or at least about 2.5 g/L. In some embodiments, the salt comprises sodium chloride (NaCl). In some embodiments, the salt comprises 25-200 mM NaCl, e.g., about 50 mM, about 75 mM, about 100 mM, about 125 mM, about 150 mM, or about 175 mM.

The present disclosure also provides a method of producing an influenza viral vaccine formulated comprising purified viral proteins, the method comprising the steps:
a. purifying viral particles from a harvested cell culture of MDCK cells;
b. inactivating and splitting the viral particles to produce sub-virions; and
c. purifying the sub-virions to produce purified viral proteins;
   wherein inactivating and splitting the viral particles of step b comprises treating the viral particles with a reagent comprising a non-ionic surfactant and a salt, and wherein the non-ionic surfactant and the salt are in an amount effective to reduce particle size of the viral particles.

The present disclosure also provides a method of producing an influenza viral vaccine formulated comprising purified viral proteins, the method comprising the steps:
a. purifying viral particles from a harvested cell culture of MDCK cells;
b. inactivating and splitting the viral particles to produce sub-virions; and
c. purifying the sub-virions to produce purified influenza viral proteins;
   wherein inactivating and splitting the viral particles of step b comprises treating the viral particles with a reagent comprising an ionic surfactant and a salt, and wherein the ionic surfactant and the salt are in an amount effective to reduce particle size of the viral particles.

The present disclosure also provides a method of producing an influenza viral vaccine formulated comprising purified viral proteins, the method comprising the steps:
a. purifying viral particles from a harvested cell culture of MDCK cells;
b. treating the purified viral particles with alkylating agent to inactivate the viral particles;
c. treating the inactivated viral particle with an ionic surfactant to split the inactivated viral particles to produce subvirions; and
d. purifying the sub-virions to produce purified influenza viral proteins;
   wherein inactivating and splitting the viral particles of step b and/or c further comprises treating the viral particles with a reagent comprising a non-ionic surfactant and an ionic surfactant, and wherein the non-ionic surfactant and the ionic surfactant are in an amount effective to reduce particle size of the viral particles.

The present disclosure also provides a method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
   wherein inactivating and splitting the viral particles of step b comprises treating the viral particles with a reagent comprising CTAB, NaCl, and polysorbate 80, in an amount effective to reduce particle size of the viral particles.

The present disclosure also provides a method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
   wherein inactivating and splitting the viral particles of step b comprises treating the viral particles with a reagent comprising 1.5-2.5 g/L CTAB, 0-150 mM NaCl, and 0-2.2 g/L polysorbate 80, in an amount effective to reduce particle size of the viral particles.

The present disclosure also provides a method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
   wherein inactivating and splitting the viral particles of step b comprises treating the viral particles with a reagent comprising 1.5-2.5 g/L of an ionic surfactant, 0-150 mM of a salt, and 0-2.2 g/L of a non-ionic surfactant, in an amount effective to reduce particle size of the viral particles.

In some embodiments, the ionic surfactant is CTAB. In some embodiments, the salt is NaCl. In some embodiments, the non-ionic surfactant is polysorbate 80.

Method of Production

Disclosed herein are processes for making a viral vaccine formulated in a sub-virion form. As used herein, the term “viral vaccine” refers to biological preparation made from a virus, that may provide immunity to a particular disease when administered to a subject. A vaccine composition may comprise one or more antigens from one or more viruses or viral strains, and will contain a sufficient amount of the antigen(s) so as to produce an immunological response in the subject. As used herein, the term “sub-virion form” means that the vaccine compositions of the disclosure will generally be formulated (a) in the form of a split virus, where the viral lipid envelope has been dissolved or disrupted, or (b) in the form of a subunit virus, comprising one or more purified viral proteins. The process of forming sub-virions, or “splitting” are described herein.

Growth in Culture

The viral vaccine disclosed herein may be produced from viruses grown on eggs or in cell culture. In some embodiments, viruses as used herein are grown in specific pathogen-free (SPF) embryonated hen eggs, and purified from the egg contents (allantoic fluid). Without being bound by theory, viral vaccines produced from viruses grown in animal cell cultures may give rise to fewer allergic reactions. Exemplary animal cell lines that may be used to produce viruses used herein include, but are not limited to, hamster, cattle, primate (including humans and monkeys) and dog cells. In some embodiments, viruses as used herein are grown in animal cell culture. In some embodiments, viruses as used herein are grown in mammalian cell culture. Exemplary mammalian cell types that may be used to produce viruses used herein include, but are not limited to, kidney cells, fibroblasts, retinal cells, and lung cells. The original MDCK cell line, available from the American Type Culture Collection (ATCC) as CCL 34, or any of its derivative cell lines may be used. In some embodiments, viruses used herein are grown in MDCK cell. For growth on a cell line, such as on MDCK cells, virus may be grown on cells in suspension or in adherent culture. One suitable MDCK cell line for suspension culture is MDCK 33016 (deposited as DSM ACC 2219). Additional examples of suitable cell lines as well as cell culturing methods can be found in WO 2007/052055, which is incorporated herein by reference in its entirety. Methods of purifying viruses, e.g., influenza viruses, from the cell culture are well known in the art.

The methods and compositions disclosed herein may be used to produce viral vaccines from any virus known in the art. Exemplary viruses include but are not limited to adenovirus, influenza virus, herpes virus, hepatitis virus, human papillomavirus, rubeola virus, mumps virus, poliomyelitis virus, Japanese encephalitis virus, rabies virus, rubella virus, Variola virus, Varicella-Zoster virus, and yellow fever virus. In some embodiments, the viral particles are from the influenza virus. In some embodiments, the viral particles are from the influenza virus A strain. In some embodiments, the viral particles are from the influenza virus B strain.

Inactivating and Splitting Viruses

As used herein, the term “inactivating” refers to the process of rendering a virus inactive, unable to replicate, and/or unable to infect, by chemical or surface alteration of viral components. Methods of inactivating viruses, such as influenza viruses, are well known in the art, e.g., see Budowsky et al., Vaccine, (1999) 9(6):398-402; WO2011/138229; and WO2011/138682. Exemplary methods of inactivating viruses include, but are not limited to pasteurization, treatment with dry heat, vapor heat, solvent/detergent, alkylating agent and/or low pH. Without being bound by theory, alkylating agents may modify viral proteins thereby inhibiting viral cell entry or the release of the viral genome. Alkylating agents may also permeate the protein capsid of viruses and chemically inactivate the viral nucleic acids. Exemplary alkylating agents that may be used to inactivate viruses for use herein include, but are not limited to formalin, β-propiolactone (β-PL), and N-acetyl-aziridine. In some embodiments, inactivation of viral particles is performed by treating the viral particles with an alkylating agent. In some embodiments, inactivation of viral particles is performed by treating the viral particles with β-propiolactone.

As used herein, the term “splitting” refers to disrupting or fragmenting whole virus, whether infectious (wild-type or attenuated) or non-infectious (e.g. inactivated), with a disrupting concentration of a splitting agent. Without being bound by theory, splitting agents generally include agents capable of breaking up and dissolving lipid membranes, typically with a hydrophobic tail attached to a hydrophilic head. Disruption may result in a full or partial solubilization of the virus proteins, thereby altering the integrity of the virus.

Methods of splitting viruses, such as influenza viruses, are well known in the art e.g. see WO02/28422, WO02/067983, WO02/074336, WO01/21151, etc. Splitting of viral particles may comprise treating the viral particles with one or more of a non-ionic surfactant and/or an ionic (e.g. cationic) surfactant. As used herein, a “surfactant” refers to an agent that is capable of lowering the surface tension (or interfacial tension) between two phases. Surfactants may act as detergents, wetting agents, emulsifiers, foaming agents, and dispersants. Without being bound by theory, surfactants are generally amphiphilic with a hydrophobic “head” and one or two hydrophilic “tails.” As used herein, the term “non-ionic surfactant” refers to surfactants with no charged groups on its head, while the term “ionic surfactant” refers to surfactants with a net positive (cationic) or net negative (anionic) charge on its head. Exemplary non-ionic surfactants include, but are not limited to alkylglycosides, alkylthioglycosides, acyl sugars, polyoxyethylene sorbitan esters (e.g, polysorbate 20 or Tween 20, polysorbate 40, polysorbate 60 or polysorbate 80), the octyl- or nonylphenoxy polyoxyethanols (e.g. the Triton surfactants, such as Triton X-100 or Triton N101), polyoxyethylene ethers, polyoxyethylene esters, polyoxyethylene alkyl ethers, Hecameg, N,N-dialkyl-Glucamides, and alkyl phenoxy polyethoxy ethanols. Exemplary ionic surfactants include, but are not limited to sulphobetaines, betaines, sarcosyl, quaternary ammonium compounds, e.g., CTAB (cetyl trimethyl ammonium bromide), Cetrimide (myristyltrimethylammonium bromide), lipofectin, lipofectamine, and DOT-MA.

In some embodiments, splitting the viral particles comprises treating the viral particles with a reagent comprising a non-ionic surfactant. In some embodiments, the non-ionic surfactant comprises polysorbate 80. In some embodiments, the non-ionic surfactant comprises about 0.2, about 0.4, about 0.6, about 0.8, about 1.0, about 1.2, about 1.4, about 1.6, about 1.8, about 2.0, about 2.2, about 2.4, about 2.6, about 2.8, or about 3.0 g/L polysorbate 80. In some embodiments, the non-ionic surfactant comprises at least about 1.0 g/L polysorbate 80, e.g., at least about 1.5 g/L, at least about 2.0 g/L, or at least about 2.5 g/L. In some embodiments, the non-ionic surfactant comprises about 0.0 g/L-2.2 g/L polysorbate 80. In some embodiments, splitting the viral particles comprises treating the viral particles with a reagent comprising an ionic surfactant. In some embodiments, the ionic surfactant comprises cetyl trimethyl ammonium bromide or cetrimonium bromide (CTAB). In some embodiments, the ionic surfactant comprises about 1.25 g/L-3.0 g/L CTAB, e.g., about 1.5-2.5 g/L, about 2.0 g/L, about 2.5 g/L, or about 3.0 g/L CTAB.

Splitting of viral particles may also comprise treating the viral particles with one or more salts. Without being bound by theory, a salt may stabilize viral particle and sub-virions. Exemplary salts that may be used include but are not limited to sodium chloride, potassium chloride, magnesium chloride, potassium phosphate, calcium phosphate. In some embodiments, splitting the viral particles comprises treating the viral particles with a reagent comprising a salt. In some embodiments, the salt is NaCl. In some embodiments, the salt comprises 0-200 mM NaCl, e.g., about 25 mM, about 50 mM, about 75 mM, about 100 mM, about 125 mM, about 150 mM, or about 175 mM NaCl. In some embodiments, the non-ionic surfactant, the ionic surfactant and/or the salt are each present in an amount effective to reduce particle size of the viral particles.

Inactivating and splitting the viral particles may also comprise adding enzymes that advantageously degrade matrix protein and reduce sub-virion particle size. Exemplary enzymes include but are not limited to proteases (e.g., proteinase K, trypsin, pepsin, elastase, thrombin, chymotrypsin, papin etc.) and nucleases (e.g., benzonase, RNAse A, RNAse H, etc.). In some embodiments, inactivating and splitting the viral particles further comprises adding a protease. In some embodiments, the protease is trypsin. In some embodiments, the protease is proteinase K. In some embodiments, inactivating and splitting the viral particles further comprises adding a nuclease. In some embodiments, the protease is benzonase.

Purifying the Split Virions

Methods of purifying sub-virions, individual proteins or antigens from viruses are well known to persons of ordinary skill in the art, and include, for example, filtration, chromatography, centrifugation, ultrafiltration and diafiltration. In some embodiments, the sub-virions are purified by size-exclusion chromatography (SEC). In some embodiments, the sub-virions are purified by ultracentrifugation. In some embodiments, the sub-virions are purified by adsorption filtration. In some embodiments, the sub-virions are purified by adsorption filtration with a polymer resin. In some embodiments, the sub-virions are purified by ultrafiltration or diafiltration. In some embodiments, the sub-virions are purified by one or more of ultracentrifugation, adsorption filtration, ultrafiltration, and diafiltration.

The methods and compositions disclosed herein, e.g., with a non-ionic surfactant, an ionic surfactant and a salt, may advantageously produce viral vaccines formulated in a sub-virion form that have a reduced particle size as compared to that produced without the surfactants or salt. Viral particles with a reduced particle size may result in improved filtration throughput and higher yield. In some embodiments, adsorption filtration throughput is at least about 5 L/m², e.g., at least about 10 L/m², at least about 15 L/m², or at least about 20 L/m². In alternative, large scale production embodiments of the present disclosure adsorption throughput is at least about 50L/m² to 350L/m². For example, at least about 50L/m², at least about 100L/m², at least about 200L/m², at least about 300L/m², or at least about 350L/m².

Methods of measuring viral particle size are well known to persons of skill in the art, and include, but are not limited to small-angle X-ray scattering (SAXS), dynamic light scattering (DLS), resonant mass measurement, size-exclusion chromatography (SEC) and laser diffraction. While other particle sizing techniques such as liquid chromatography may be unsuitable due to intrusive sample preparation, undesirable surface chemistry of the column-resin interacting with test samples, or high column pressure that could potentially alter high-order structures, DLS is a rapid, non-contact, nonintrusive particle sizing technique that can test analytes in their native state without compromising the integrity of high-order structures. In some embodiments, viral particle size is measured by dynamic light scattering (DLS). In some embodiments, virus and sub-virion particle size is measured by SAXS. In some embodiments, viral and sub-virion particle size is measured by resonant mass measurement. In some embodiments, viral and sub-virion particle size is measured by size exclusion chromatography (SEC). In some embodiments, particle size is defined by the hydrodynamic radius (R_{H}) of the viral particle. In some embodiments, particle size is defined by the radius of gyration (R_{G}) of the viral particle. In some embodiments, the particle size of the sub-virion is less than about 500 nm, e.g., less than about 400 nm, less than about 300 nm, less than about 200 nm, or less than about 150 nm.

The present disclosure is further illustrated by the following examples that should not be construed as limiting. The contents of all references, patents, and published patent applications cited throughout this application, as well as the Figures, are incorporated herein by reference in their entirety for all purposes.

### EXAMPLES

The following examples are to be considered illustrative and not limiting on the scope of the disclosure disclosed above

### Example 1

Method: Purifying Viral Particles from Harvested Cell Culture

Clarified harvest from MCDK cell cultures comprising the influenza virus was concentrated and diafiltered using hollow fiber membranes. The clarified harvest was filled into the retentate vessel of an ultrafiltration tank (UFO) and concentrated to a target volume, then diafiltered against a salt-containing Tris buffer. After diafiltration, the retentate was concentrated again and then flushed with the same buffer to recover the product. The product was then processed in a chromatography step to remove host cell proteins (HCPs) and purify virus particles. The concentrated and diafiltered cell harvest was loaded onto the column through pre-equilibrated filters. The product was collected in the flow through and added into the retentate vessel of an ultrafiltration tank (UF1) with hollow fiber membranes. The product was concentrated to a target volume, then diafiltered at a constant volume with a buffering containing MgCl₂. Benzonase solution was added and the product was then recirculated through the ultrafilter to allow digestion of DNA. Following the digestion, a second diafiltration was performed at a constant volume with sodium phosphate to exchange the product into the right buffer for inactivation.

Method: Inactivating and Splitting Viral Particles

Polysorbate 80 (PS80) in sodium phosphate was added to the product from UF1, and the mixture was incubated at room temperature. The influenza virus was then inactivated using b-propiolactone (BPL). Briefly, the virus and PS80 mixture was cooled to 5° C. before BPL was added for inactivation of the influenza virus. Following inactivation, the mixture was warmed to 37° C. to hydrolyze BPL. The mixture was then brought to room temperature prior to addition of PS80, as discussed below.

Additional PS80 was then added to the mixture at a concentration as shown in Table 1. Sodium chloride was then added at a concentration as shown in Table 1. Finally, CTAB was added at a concentration as shown in Table 1, then incubated at room temperature and then transferred to an ultracentrifuge.

Method: Purifying the Sub-Virions

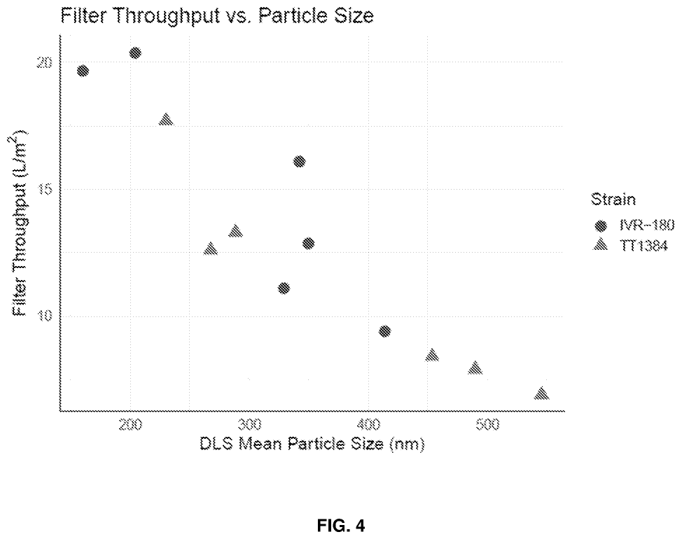

The solubilized surface antigens were separated from the viral cores by continuous ultracentrifugation. The ultracentrifuge was flushed with sodium phosphate to enhance product recovery. The flow-through containing viral antigens was collected in an adsorption vessel. PS80 was added to make a final concentration of 2.5 g/L. A polymeric resin in sodium phosphate was then added and incubated with the mixture for a sufficient time for adsorption of CTAB to take place. After incubation, the product was removed from the resin and filtered through a 0.5/0.2 μm filter before ultrafiltration. Overall product recovery yield from the adsorption filter was measured by throughput as shown in FIG. 4.

TABLE 1
Concentrations of PS80, NaCl and CTAB used for splitting

| | | | | Shown in |
|---|---|---|---|---|
| Condition | PS80 (g/L) | NaCl (mM) | CTAB (g/L) | FIGS. |
| 1 | 0 | 0 | 1.5 | 1A, 2A, 3A |
| 2 | 0 | 0 | 2.0 | 1B, 2B, 3B |
| 3 | 0 | 0 | 2.5 | 1C, 2C, 3C |
| 4 | 0 | 75 | 1.5 | 1A, 2A, 3A |
| 5 | 0 | 75 | 2.0 | 1B, 2B, 3B |
| 6 | 0 | 75 | 2.5 | 1C, 2C, 3C |
| 7 | 0 | 150 | 1.5 | 1A, 2A, 3A |
| 8 | 0 | 150 | 2.0 | 1B, 2B, 3B |
| 9 | 0 | 150 | 2.5 | 1C, 2C, 3C |
| 10 | 1.1 | 0 | 1.5 | 1A, 2A, 3A |
| 11 | 1.1 | 0 | 2.0 | 1B, 2B, 3B |
| 12 | 1.1 | 0 | 2.5 | 1C, 2C, 3C |
| 13 | 1.1 | 75 | 1.5 | 1A, 2A, 3A |
| 14 | 1.1 | 75 | 2.0 | 1B, 2B, 3B |
| 15 | 1.1 | 75 | 2.5 | 1C, 2C, 3C |
| 16 | 1.1 | 150 | 1.5 | 1A, 2A, 3A |
| 17 | 1.1 | 150 | 2.0 | 1B, 2B, 3B |
| 18 | 1.1 | 150 | 2.5 | 1C, 2C, 3C |
| 19 | 2.2 | 0 | 1.5 | 1A, 2A, 3A |
| 20 | 2.2 | 0 | 2.0 | 1B, 2B, 3B |
| 21 | 2.2 | 0 | 2.5 | 1C, 2C, 3C |
| 22 | 2.2 | 75 | 1.5 | 1A, 2A, 3A |
| 23 | 2.2 | 75 | 2.0 | 1B, 2B, 3B |
| 24 | 2.2 | 75 | 2.5 | 1C, 2C, 3C |
| 25 | 2.2 | 150 | 1.5 | 1A, 2A, 3A |
| 26 | 2.2 | 150 | 2.0 | 1B, 2B, 3B |
| 27 | 2.2 | 150 | 2.5 | 1C, 2C, 3C |

Results

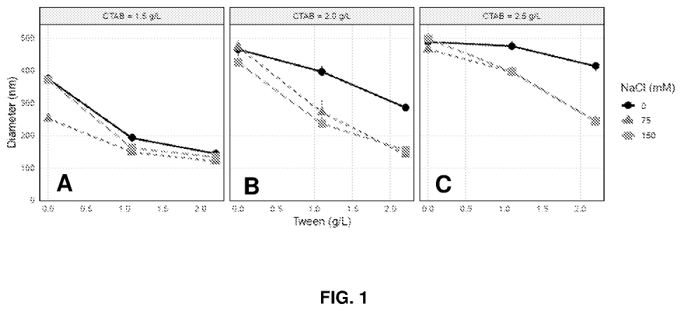

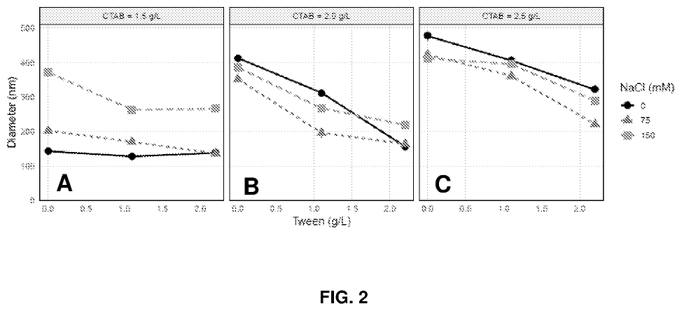

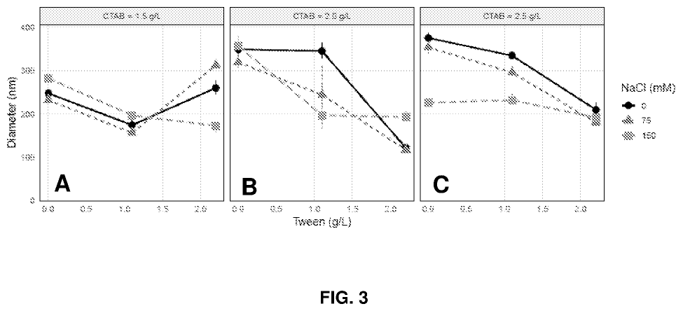

The study was performed as detailed in the method sections above, on three different influenza virus samples: (1) Influenza B/Victoria lineage (shown in FIGS. 1A-C), (2) Influenza A H3N2 from Maine (shown in FIGS. 2A-C), and (3) Influenza A H3N2 from South Carolina (shown in FIGS. 3A-C). The average particle size measured by DLS at different conditions for the three samples are shown in FIGS. 1A-C, 2A-C, and 3A-C respectively. Error bars indicate the range in repeated measurements. As shown in FIGS. 1A-C, 2A-C and 3A-C, lower levels of CTAB, and higher levels of either polysorbate 80 generally produces sub-virions that have smaller particle sizes.

### Example 2

### Particle Size Determination by Dynamic Light Scattering

Particle size of the viral particles in sub-virion form was measured using dynamic light scattering (DLS) on a Wyatt Technologies DynaPro Plate Reader II instrument. Before and during the measurement, dust was removed with a 0.1 μm anotop syringe filter or by centrifuging samples in conical tubes at 3,000 rpm for 10 minutes on a fixed angle benchtop centrifuge. A reference standard of 14 kDa lysozyme at 0.25 mg/mL in aqueous solution was used as a benchmark. The samples and reference standards were loaded onto a 384-well glass bottom microplate (Greiner P/N 781892) and the microplate was spun at 2,000×g for 2 minutes in a swinging-bucket centrifuge to dissipate bubbles. The microplate was loaded into the DLS instrument and 14 data acquisitions at 25 seconds acquisition time each was performed at 25° C.

Data analysis was done by the DYNAMICS software package to generate sum-of-squares difference from the Gaussian cumulant results and regularized results were reported. A single Gaussian distribution of correlation rates was used during curve-fitting to generate monomodal analyses for results with ≤57% polydispersity. Results exhibiting low sum-of-squares values ( [symbol] 20) indicated reasonable mathematical agreement between the measured correlation curve and the cumulants fitted curve, and suggested that the sample was likely monomodal with low polydispersity, specifically a tight size distribution. Results that exhibited SOS>20 and/or % polydispersity>57% were reported as multimodal and results from the Regularized Graphs were reported.

The results of Experiment 2 are shown in FIG. 4, where Filter throughput versus DLS mean particle size was measured and compared. As shown, as the particle size reduces overall throughput through the filters increases, which results in an increased yield.

### Example 3

### Adsorption Filtration Experiments with A/Turkey/Turkey Strain

Adsorption filtration experiments were conducted, and four splitting conditions were tested using A/turkey/Turkey/1/2005 NIBRG-23 (H5N1). More particularly, the splitting conditions tested were for adsorption filterability with various concentrations of CTAB, NaCl, and PS80, as shown in Table 2 below. A follow-up adsorption filtration experiment for A/turkey/Turkey/1/2005 NIBRG-23 (H5N1) was conducted, as shown in Table 3 below.

TABLE 2
Splitting Conditions and Filterability Results

| Filtration | | | | Average Filter |
|---|---|---|---|---|
| Condition | CTAB (g/L) | NaCl (mM) | PS80 (g/L) | throughput (L/m²) |
| 1a | 1.6 | 150 | 0.2 | 3.53 |
| 1b | 1.6 | 150 | 0.2 | 3.87 |
| 2a | 2.5 | 150 | 0.2 | 3.57 |
| 2b | 2.5 | 150 | 0.2 | 4.53 |
| 3a | 2.5 | 80 | 1.2 | 5.37 |
| 3b | 2.5 | 80 | 1.2 | 10.9 |
| 4a | 2.5 | 24 | 2.2 | 254 |
| 4b | 2.5 | 24 | 2.2 | 138 |

The results from Table 2 suggest that higher concentrations of CTAB and PS80 and lower concentrations of NaCl exhibit optimal filtration performance (see, e.g., Filtration Conditions 4a and 4b) for the A/turkey/Turkey (H5N1) Strains. Splitting conditions were identified that produced throughput 36-72 times better than existing conditions.

TABLE 3
Filterability Results

| Filtration | CTAB | NaCl | PS80 | Supernatant | Average Filter | |
|---|---|---|---|---|---|---|
| Condition | (g/L) | (mM) | (g/L) | Turbidity (NTU) | throughput (L/m²) | Vmax (L/m²) |
| 1 | 2.5 | 0 | 2.2 | 15 | 62 | 104 |
| 2 | 2.5 | 25 | 2.2 | 16 | 76 | 93 |
| 3 | 2.5 | 50 | 2.2 | 22 | 31 | 36 |
| 4 | 2.5 | 75 | 2.2 | 44 | 12 | 13 |

The results from Table 3 suggest that, at constant concentrations of 2.5 g/L CTAB and 2.2 g/L PS80, increasing NaCl correlates with increased supernatant turbidity and decreased filter throughput. Therefore, decreased NaCl appears to provide significant improvement in filterability for the A/turkey/Turkey (H5N1) strain. At constant CTAB (2.5 g/L) and constant PS80 (2.2 g/L), filterability (throughput) improved about seven-fold as NaCl was decreased from 75 mM to zero.

TABLE 4
Summary of the Filterability Results from Tables 2 and 3

| | | | | | | DOE | | | Adsorption |
|---|---|---|---|---|---|---|---|---|---|
| | | | | | SP1 | Condition | | | Filtration |
| | CTAB | NaCl | PS80 | H1 | (SRD/ | Most | NA by | SDS- | Throughput |
| Condition | (g/L) | (mM) | (g/L) | (SRD) | BCA) | Similar | Western | PAGE | (L/m²) |
| Previous | 1.6 | 150 | 0.2 | 571 | 0.83 | 7 | faint | Some MP | 3.87 |
| Recom'd | | | | | | | | and NP | |
| Condition | | | | | | | | | |
| Yield & SP | 2.5 | 75 | 2.2 | 637 | 0.87 | 24 | present | Some MP | 12 |
| Optimum | | | | | | | | and NP | |
| Filtration | 2.5 | 0 | 2.2 | 398 | 0.68 | 21 | present | No MP | 82 |
| Optimum | | | | | | | | and NP | |
| | | | | | | | | observed | |

| | | | | | | | | |
|---|---|---|---|---|---|---|---|---|
| Recom- | 2.5 | 25 | 2.2 | 502 | 0.77 | 21&24 | Not tested, but | 254; 76* |
| mended | | | | | | more like | predicted to be | |
| Condition | | | | | | 21 | intermediate | |
| | | | | | | | between 21 and 24 | |

### Example 4

Method: Purifying Viral Particles from Harvested Cell Culture Under Large Scale Manufacturing Conditions

Clarified harvest from MCDK cell cultures comprising the influenza virus is concentrated and diafiltered using hollow fiber membranes against a salt-containing Tris buffer. The harvest product is then further concentrated, and the system flushed with the same buffer to recover the product. This material is passed through a 1.2 μm column guard filter and applied to a chromatography column for removal of HCP to arrive at purified whole virus. The purified whole virus is concentrated and diafiltered (UF/DF1) into a magnesium containing buffer using hollow fiber membranes. Benzonase is added to remove DNA. A second diafiltration is performed to exchange the purified whole virus into a phosphate buffer and the system flushed with the same buffer to recover the product. The purified whole virus is inactivated in the presence of Polysorbate 80 and BPL at 2-8° C. Following inactivation, the BPL is hydrolyzed at 37° C. Virus splitting occurs with the strain specific addition of Polysorbate 80, sodium chloride, and CTAB to solubilize the surface antigens within the ranges of 1.0-2.5 g/L, 0-200 mM, and 1.25-3.0 g/L, respectively, or alternatively as shown in Table 1. Viral cores are removed by continuous flow ultracentrifugation and the soluble fraction is contacted with a polymeric resin to remove CTAB. The resin is removed by passing the product slurry through a 60 μm mesh bag and the soluble viral surface antigens are then processed through a 0.5/0.2 μm filter. The filtered antigens are concentrated and diafiltered (UF/DF2), using cassette membranes, into the final formulation buffer prior to processing through a 0.2 μm filter.

1. A method of producing a viral vaccine formulated in a sub-virion form, the method comprising the steps:
a. purifying viral particles from harvested cell culture;
b. inactivating and splitting the viral particles to produce sub-virions; and
c. purifying the sub-virions;
wherein inactivating and splitting the viral particles of step b comprises treating the viral particles with a reagent comprising a non-ionic surfactant and an ionic surfactant, and wherein the at non-ionic surfactant and the ionic surfactant is present in an amount effective to reduce particle size of the viral particles.

2. The method of claim 1, wherein the reagent further comprises a salt, wherein the salt is present in an amount effective to reduce particle size of the viral particles.

3. The method of any one of claims 1-2, wherein the average hydrodynamic radius of the sub-virion ranges from 150 nm to 350 nm.

4. The method of any one of claim 1-3, wherein the non-ionic surfactant is polysorbate 80.

5. The method of any one of claims 1-4, wherein the non-ionic surfactant comprises at least about 1.0 g/L polysorbate 80, e.g., at least about 1.5 g/L, at least about 2.0 g/L, or at least about 2.5 g/L.

6. The method of any one of claims 1-5, wherein the ionic surfactant comprises cetrimonium bromide (CTAB).

7. The method of any one of claims 1-6, wherein the ionic surfactant comprises about 1.25 g/L-3.0 g/L CTAB, e.g., about 1.5 g/L, about 2.0 g/L, about 2.5 g/L, or about 3.0 g/L.

8. The method of any one of claims 1-7, wherein the salt comprises sodium chloride (NaCl).

9. The method of any one of claims 1-8, wherein the salt comprises 0-200 mM NaCl, e.g., about 25 mM, about 50 mM, about 75 mM, about 100 mM, about 125 mM, about 150 mM, or about 175 mM.

10. The method of any one of claims 1-9, wherein the non-ionic surfactant comprises polysorbate 80, the ionic surfactant comprises CTAB, and the salt comprises sodium chloride NaCl.

11. The method of any one of claims 1-10, wherein purifying in step c comprises adsorption filtration.

12. The method of claim 11, wherein adsorption filtration throughput is at least about 50 L/m², e.g., at least about 100 L/m², at least about 150 L/m², at least about 200 L/m², at least about 200 L/m², at least about 300 L/m², or at least about 350 L/m².

13. The method of any one of claims 1-12, wherein the particle size of the sub-virion is less than about 500 nm, e.g., less than about 400 nm, less than about 300 nm, less than about 200 nm, or less than about 150 nm.

14. The method of claim 1-13, wherein the particle size of the sub-virion is the hydrodynamic radius.

15. The method of any one of claims 1-14, wherein the size of the sub-virion is measured by dynamic light scattering (DLS).

16. The method of any one of claims 1-15, wherein the viral particles are from the influenza virus.

17. The method of any one of claims 1-16, wherein the viral particles are from the influenza virus A strain.

18. The method of any one of claims 4-17, wherein the concentration of polysorbate 80 is 0.3 g/L prior to splitting.

19. The method of any one of claims 4-18, wherein the concentration of polysorbate 80 increases at a range of 0-2.2 g/L during splitting.

20. The method of any one of claims 4-19, wherein the concentration of polysorbate 80 is 0.3 g/L, 1.4 g/L, or 2.5 g/L during splitting.

21. The method of any one of claims 8-20, wherein a concentration of NaCl ranging from 0-25 mM is associated with increased filtration throughput.

22. A method of reducing viral particle size, the method comprising treating the viral particles with a reagent comprising a non-ionic surfactant and an ionic surfactant, and wherein the non-ionic surfactant and the ionic surfactant are present in an amount effective to reduce particle size of the viral particles.

23. The method of claim 22 wherein the reagent further comprises a salt, wherein the salt is present in an amount effective to reduce particle size of the viral particles.

24. The method of any one of claims 22-23, wherein the non-ionic surfactant comprises polysorbate 80.

25. The method of any one of claims 22-24, wherein the non-ionic surfactant comprises at least about 1.0 g/L polysorbate 80, e.g., at least about 1.5 g/L, at least about 2.0 g/L, or at least about 2.5 g/L.

26. The method of any one of claims 22-25, wherein the ionic surfactant comprises cetrimonium bromide (CTAB).

27. The method of any one of claims 22-26, wherein the ionic surfactant comprises about 1.25 g/L-3.0 g/L CTAB, e.g., about 1.5 g/L, about 2.0 g/L, about 2.5 g/L, or about 3.0 g/L.

28. The method of any one of claims 22-27, wherein the salt comprises sodium chloride (NaCl).

29. The method of any one of claims 22-28, wherein the salt comprises 25-200 mM NaCl, e.g., about 50 mM, about 75 mM, about 100 mM, about 125 mM, about 150 mM, or about 175 mM.

30. The method of any one of claims 22-29, wherein the non-ionic surfactant comprises polysorbate 80, the ionic surfactant comprises CTAB, and the salt comprises sodium chloride NaCl.

31. The method of any one of claims 22-30, further comprising filtering the treated viral particles through an adsorption filter.

32. The method of claim 31, wherein the throughput of the adsorption filter is at least about 50 L/m², e.g., at least about 100 L/m², at least about 150 L/m², at least about 200 L/m², at least about 250 L/m², at least about 300 L/m², or at least about 350 L/m².

33. The method of any one of claims 22-32, wherein the viral particle size is less than about 500 nm, e.g., less than about 400 nm, less than about 300 nm, less than about 200 nm, or less than about 150 nm.

34. The method of claim 22-33, wherein the viral particle size is the hydrodynamic radius of the viral particle.

35. The method of any one of claims 22-34, wherein the viral particle size is measured by dynamic light scattering (DLS).

36. The method of any one of claims 22-35, wherein the viral particles are from the influenza virus.

37. The method of any one of claims 22-36, wherein the viral particles are from the influenza virus A strain.

38. The method of any one of claims 24-37, wherein the concentration of polysorbate 80 is 0.3 g/L prior to splitting.

39. The method of any one of claims 24-38, wherein the concentration of polysorbate 80 increases at a range of 0-2.2 g/L during splitting.

40. The method of any one of claims 24-39, wherein the concentration of polysorbate 80 is 0.3 g/L, 1.4 g/L, or 2.5 g/L during splitting.

41. The method of any one of claims 28-40, wherein a concentration of NaCl ranging from 0-25 mM is associated with increased filtration throughput.

42. A method of reducing viral particle size in an influenza virus purification process, the method comprising
a. purifying viral particles harvested from cell culture;
b. inactivating and splitting the viral particles to produce sub-virions; and
c. purifying the sub-virions;
wherein activating and splitting the viral particles of step b comprises treating the viral particles with a reagent comprising a non-ionic surfactant and an ionic surfactant, and wherein the non-ionic surfactant and the ionic surfactant are present in an amount effective to reduce particle size of the viral particles.

43. A method of producing a viral vaccine formulated in a sub-virion form, the method comprising the steps:
a. purifying viral particles from harvested cell culture;
b. inactivating and splitting the viral particles to produce sub-virions; and
c. purifying the sub-virions;
wherein activating and splitting the viral particles of step b comprises treating the viral particles with a reagent comprising a non-ionic surfactant and an ionic surfactant, and wherein the non-ionic surfactant and the ionic surfactant are present in an amount effective to reduce particle size of the viral particles.

44. A method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
wherein inactivating and splitting the viral particles of step b comprises treating the viral particles with a reagent comprising a non-ionic surfactant and an ionic surfactant, and wherein the non-ionic surfactant and the ionic surfactant are present in an amount effective to reduce particle size of the viral particles.

45. The method of any one of claims 42-44 wherein the reagent further comprises a salt, wherein the salt is present in an amount effective to reduce particle size of the viral particles.

46. The method of any one of claims 42-45, wherein the non-ionic surfactant comprises polysorbate 80.

47. The method of any one of claims 42-46, wherein the non-ionic surfactant comprises at least about 1.0 g/L polysorbate 80, e.g., at least about 1.5 g/L, at least about 2.0 g/L, or at least about 2.5 g/L.

48. The method of any one of claims 42-47, wherein the salt comprises sodium chloride (NaCl).

49. The method of any one of claims 42-48, wherein the salt comprises 25-200 mM NaCl, e.g., about 50 mM, about 75 mM, about 100 mM, about 125 mM, about 150 mM, or about 175 mM.

50. The method of any one of claims 46-49, wherein the concentration of polysorbate 80 is 0.3 g/L prior to splitting.

51. The method of any one of claims 46-50, wherein the concentration of polysorbate 80 increases at a range of 0-2.2 g/L during splitting.

52. The method of any one of claims 46-51, wherein the concentration of polysorbate 80 is 0.3 g/L, 1.4 g/L, or 2.5 g/L during splitting.

53. The method of any one of claims 48-52, wherein a concentration of NaCl ranging from 0-25 mM is associated with increased filtration throughput.

54. A method of producing an influenza viral vaccine formulated comprising purified viral proteins, the method comprising the steps:
a. purifying viral particles from a harvested cell culture of MDCK cells;
b. inactivating and splitting the viral particles to produce sub-virions; and
c. purifying the sub-virions to produce purified viral proteins;
wherein inactivating and splitting the viral particles of step b comprises treating the viral particles with a reagent comprising a non-ionic surfactant and a salt, and wherein the non-ionic surfactant and the salt are in an amount effective to reduce particle size of the viral particles.

55. A method of producing an influenza viral vaccine formulated comprising purified viral proteins, the method comprising the steps:
a. purifying viral particles from a harvested cell culture of MDCK cells;
b. inactivating and splitting the viral particles to produce sub-virions; and
c. purifying the sub-virions to produce purified influenza viral proteins;
wherein inactivating and splitting the viral particles of step b comprises treating the viral particles with a reagent comprising an ionic surfactant and a salt, and wherein the ionic surfactant and the salt are in an amount effective to reduce particle size of the viral particles.

56. A method of producing an influenza viral vaccine formulated comprising purified viral proteins, the method comprising the steps:
a. purifying viral particles from a harvested cell culture of MDCK cells;
b. treating the purified viral particles with alkylating agent to inactivate the viral particles;
c. treating the inactivated viral particle with an ionic surfactant to split the inactivated viral particles to produce subvirions; and
d. purifying the sub-virions to produce purified influenza viral proteins;
wherein inactivating and splitting the viral particles of step b and/or c further comprises treating the viral particles with a reagent comprising a non-ionic surfactant and an ionic surfactant, and wherein the non-ionic surfactant and the ionic surfactant are in an amount effective to reduce particle size of the viral particles.

57. A method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
wherein inactivating and splitting the viral particles of step b comprises treating the viral particles with a reagent comprising CTAB, NaCl, and polysorbate 80, in an amount effective to reduce particle size of the viral particles.

58. The method of claim 57, wherein the concentration of polysorbate 80 is 0.3 g/L prior to splitting.

59. The method of any one of claims 57-58, wherein the concentration of polysorbate 80 increases at a range of 0-2.2 g/L during splitting.

60. The method of any one of claims 57-59, wherein the concentration of polysorbate 80 is 0.3 g/L, 1.4 g/L, or 2.5 g/L during splitting.

61. The method of any one of claims 57-60, wherein a concentration of NaCl ranging from 0-25 mM is associated with increased filtration throughput.

62. A method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
wherein inactivating and splitting the viral particles of step b comprises treating the viral particles with a reagent comprising 1.5-2.5 g/L CTAB, 0-150 mM NaCl, and 0-2.2 g/L polysorbate 80, in an amount effective to reduce particle size of the viral particles.

63. A method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
wherein inactivating and splitting the viral particles of step b comprises treating the viral particles with a reagent comprising 1.5-2.5 g/L of an ionic surfactant, 0-150 mM of a salt, and 0-2.2 g/L of a non-ionic surfactant, in an amount effective to reduce particle size of the viral particles.

64. The method of claim 63, wherein the ionic surfactant is CTAB.

65. The method of any one of claims 63-64, wherein the salt is NaCl.

66. The method of any one of claims 63-65, wherein the non-ionic surfactant is polysorbate 80.

67. The method of claim 66, wherein the concentration of polysorbate 80 is 0.3 g/L prior to splitting.

68. The method of any one of claims 66-67, wherein the concentration of polysorbate 80 increases at a range of 0-2.2 g/L during splitting.

69. The method of any one of claims 66-68, wherein the concentration of polysorbate 80 is 0.3 g/L, 1.4 g/L, or 2.5 g/L during splitting.

70. The method of any one of claims 65-69, wherein a concentration of NaCl ranging from 0-25 mM is associated with increased filtration throughput.

71. A method of producing an influenza viral vaccine formulated in a sub-virion form, the method comprising the steps:
a. purifying viral particles from a harvested cell culture of MDCK cells;
b. inactivating and splitting the viral particles to produce sub-virions; and
c. purifying the sub-virions;
wherein inactivating and splitting the viral particles of step b comprises treating the viral particles with a reagent comprising CTAB, NaCl, and polysorbate 80, in an amount selected from the group consisting of conditions 1 through 27 of Table 1.
